(19) United States
(12) Patent Application Publication
(10) Pub. No.: US 2005/0154412 A1
(43) Pub. Date: Jul. 14, 2005

(54) Airway implant and delivery tool

(75) Inventors: Kurt D. Krueger, Stacy, MN (US); John P. Sopp, Forest Lake, MN (US); Brian J. Erickson, Woodbury, MN (US); John R. Frigstad, St. Anthony, MN (US); Larry A. Walter, Minneapolis, MN (US)

Correspondence Address: MERCHANT & GOULD PC, P.O. BOX 2903, MINNEAPOLIS, MN, 55402-0903, US

(73) Assignee: Restore Medical, Inc.

(21) Appl. No.: 11/066,960

(22) Filed: Feb. 25, 2005

Related U.S. Application Data
(63) Continuation-in-part of application No. 10/665,941, filed on Sep. 19, 2003.
(63) Continuation-in-part of application No. 10/665,760, filed on Sep. 19, 2003, now Pat. No. 6,899,105.

(51) Int. Cl. A61B 17/34

(52) U.S. Cl. 606/185

(57) ABSTRACT

An apparatus for treating an airway condition of a patient includes a handle sized to be hand-grasped by an operator and having an actuator mechanism to be selectively actuated by said operator. A needle is fixedly connected to the handle. The needle has an axially extending bore and a distal tip for penetrating into the airway tissue. An implant is disposed within said bore at the distal tip. The implant is formed of biocompatible material and is sized to be embedded within a tissue of said airway. The implant is ejected from the distal tip upon actuation of the actuator. The actuator has an indicator for indicating a position of the actuator and a lock for preventing movement of the actuator opposite a deployment direction.

### I. CROSS-REFERENCE TO RELATED APPLICATIONS

The present application is a continuation-in-part application of commonly assigned U.S. patent application Ser. Nos. 10/665,941 and 10/665,760 both filed Sep. 19, 2003. This application claims features of a design which is the subject of a commonly assigned design patent application Ser. No. ______, titled “Implant Delivery Tool” and filed in the name of the same inventors and on the same date as the present application.

### II. BACKGROUND OF THE INVENTION

1. Field of the Invention

This invention is directed to an apparatus for treating an airway condition of a patient. More particularly, this invention is directed to an apparatus for delivering an implant into tissue of a patient's airway.

2. Description of the Prior Art

Airway conditions such as snoring and obstructive sleep apnea (OSA) have received increased scientific and academic attention. One publication estimates that up to 20% of the adult population snores habitually. Huang, et al., “Biomechanics of Snoring”, Endeavour, p. 96-100, Vol. 19, No. 3 (1995). Snoring can be a serious cause of marital discord. In addition, snoring can present a serious health risk to the snorer. In 10% of habitual snorers, collapse of the airway during sleep can lead to obstructive sleep apnea syndrome. Id.

Notwithstanding numerous efforts to address snoring and sleep apnea, effective treatments have been elusive. Such treatment may include mouth guards or other appliances worn by the snorer during sleep. However, patients find such appliances uncomfortable and frequently discontinue use (presumably adding to marital stress).

Electrical stimulation of the soft palate has been suggested to treat snoring and obstructive sleep apnea. See, e.g., Schwartz, et al., “Effects of electrical stimulation to the soft palate on snoring and obstructive sleep apnea”, J. Prosthetic Dentistry, pp. 273-281 (1996). Devices to apply such stimulation are described in U.S. Pat. Nos. 5,284,161 and 5,792,067. Such devices are appliances requiring patient adherence to a regimen of use as well as subjecting the patient to discomfort during sleep. Electrical stimulation to treat sleep apnea is discussed in Wiltfang, et al., “First results on daytime submandibular electrostimulation of suprahyoidal muscles to prevent night-time hypopharyngeal collapse in obstructive sleep apnea syndrome”, International Journal of Oral & Maxillofacial Surgery, pp. 21-25 (1999).

Surgical treatments have been employed. One such treatment is uvulopalatopharyngoplasty. In this procedure, so-called laser ablation is used to remove about 2 cm of the trailing edge of the soft palate thereby reducing the soft palate's ability to flutter between the tongue and the pharyngeal wall of the throat. The procedure is frequently effective to abate snoring but is painful and frequently results in undesirable side effects. Namely, removal of the soft palate trailing edge comprises the soft palate's ability to seal off nasal passages during swallowing and speech. In an estimated 25% of uvulopalatopharyngoplasty patients, fluid escapes from the mouth into the nose while drinking. Huang, et al., supra at 99. Uvulopalatopharyngoplasty (UPPP) is also described in Harries, et al., “The Surgical treatment of snoring”, Journal of Laryngology and Otology, pp. 1105-1106 (1996) which describes removal of up to 1.5 cm of the soft palate. Assessment of snoring treatment is discussed in Cole, et al., “Snoring: A review and a Reassessment”, Journal of Otolaryngology, pp. 303-306 (1995).

Novel treatments for snoring and sleep apnea are described in various patents commonly assigned with the present application. These include U.S. Pat. No. 6,250,307 to Conrad et al. dated Jun. 26, 2001 which describes (along with other embodiments) elongated implants for placement in the soft palate. In one embodiment, three such implants are placed in the soft palate. U.S. Pat. No. 6,578,580 to Conrad et al. dated Jun. 17, 2003 describes a needle (which may have a perforated distal tip) for delivery of an implant. The implant may be preloaded into the needle. In U.S. Pat. No. 6,523,542 to Metzger et al. dated Feb. 25, 2003, an implant is described as a sheet of felt or similar material delivered through a needle. U.S. Pat. No. 6,513,530 to Knudson et al. dated Feb. 4, 2003 describes the implant as a braid with welded ends near frayed ends. U.S. Pat. No. 6,431,174 to Knudson et al. dated Aug. 13, 2002 describes use of microbeads as implants as well as describing placement of implants in a pharyngeal wall or nasal area as well as a soft palate.

When placing implants in the tissue of a patient's airway (i.e., in soft palate, nasal or pharyngeal wall tissue), it may often be desirable to place more than one such implant (e.g., the three parallel longitudinal implants shown in the soft palate in the aforementioned U.S. Pat. No. 6,250,307). Delivery systems should be designed to minimize fabrication costs (and fabrication and assembly error) to minimize the overall cost of a procedure. Further, the design of a delivery tool for such implants should facilitate a physician's correct use of the delivery tool and accurate placement of an implant. It is an object of the present invention to provide such a delivery system.

### III. SUMMARY OF THE INVENTION

According to one aspect of the present invention, an apparatus is disclosed for treating an airway condition of a patient. The invention includes a handle sized to be hand-grasped by an operator and having an actuator mechanism to be selectively actuated by said operator. A needle is fixedly connected to the handle. The needle has an axially extending bore and a distal tip for penetrating into the airway tissue. An implant is disposed within said bore at the distal tip. The implant is formed of biocompatible material and is sized to be embedded within a tissue of said airway. The implant is ejected from the distal tip upon actuation of the actuator. The actuator has an indicator for indicating a position of the actuator and a lock for preventing movement of the actuator opposite a deployment direction.

### IV. BRIEF DESCRIPTION OF THE DRAWINGS

FIG. 1 shows, in cross-section, a naso-pharyngeal area of an untreated patient;

FIG. 2 shows a soft palate viewed through an open mouth of the untreated patient of FIG. 1;

FIG. 3 is a front view of an interior of the mouth shown in FIG. 1 and showing an area to be ablated according to a first prior art surgical procedure;

FIG. 4 is the view of FIG. 3 and showing an area to be scarred according to a second prior art surgical procedure;

FIG. 5 is a schematic representation of a spring-mass system model of the soft palate;

FIG. 6 is perspective view of an implant for use with the present invention;

FIG. 7 is a side elevation view of a prior delivery tool for delivery of the implant of FIG. 6 into the soft palate of a patient;

FIG. 8 is a side-sectional view of a distal tip of the tool of FIG. 7 cut-away to reveal the implant of FIG. 6 pre-loaded into the distal tip of the tool;

FIG. 9 is the view of FIG. 1 with the soft palate containing the implant of FIG. 6;

FIG. 10 is the view of FIG. 2 showing three implants of the type of FIG. 6 in the soft palate;

FIG. 11 is a top, rear and left side perspective view of a preferred embodiment of a delivery tool of the present invention;

FIG. 12 is a left side elevation view of delivery tool of FIG. 111 (with the opposite side being substantially identical);

FIG. 13 is a top plan view of the delivery tool of FIG. 11;

FIG. 14 is a bottom plan view of the delivery tool of FIG. 11;

FIG. 15 is a rear end elevation view of the delivery tool of FIG. 11;

FIG. 16 is a front-end elevation view of the delivery tool of FIG. 11;

FIG. 17 is an a top, rear and left side exploded perspective view of the delivery tool of FIG. 11 showing a slider and obturator in exploded;

FIG. 18 is the view of FIG. 17 with an obturator not shown exploded and showing a slider and lock in perspective view;

FIG. 19 is a top, rear and right side perspective view of the delivery tool of FIG. 11 and showing in exploded format additional components for use in shipping and storage;

FIG. 20 is an enlarged perspective view showing engagement of a slider and lock;

FIG. 21 is a left side elevation view of a handle and needle sub-assembly for use in forming the delivery tool of the present invention;

FIG. 22 is a rear elevation view of the sub-assembly of FIG. 21;

FIG. 23 is a top plan view of the sub-assembly of FIG. 21;

FIG. 24 is an enlarged view of the portion of FIG. 23 enclosed by circle 24;

FIG. 25 is an enlarged view of the portion of FIG. 22 enclosed by circle 25;

FIG. 26 is a view taken along line 26-26 in FIG. 22;

FIG. 27 is an enlarged view of the portion of FIG. 24 enclosed by circle 27;

FIG. 28 is an enlarged view of the portion of FIG. 21 enclosed by circle 28;

FIG. 29 is a view taken along line 29-29 in FIG. 28;

FIG. 30 is an enlarged view of the portion of FIG. 29 enclosed by circle 30;

FIG. 31 is a view taken along line 31-31 in FIG. 29;

FIG. 32 is an enlarged view of the portion of FIG. 31 enclosed by circle 32;

FIG. 33 is a side elevation view of a needle component used in forming the sub-assembly of FIG. 21;

FIG. 33A is a cross-sectional view of the needle of FIG. 33 and showing a loaded implant, an obturator and a slider mechanism pin;

FIG. 34 is a side sectional schematic view of mold components for forming the sub-assembly of FIG. 21;

FIG. 34A is the view of FIG. 34 after a molding process and with mold components separated to release the sub-assembly of FIG. 21;

FIG. 35 is a top, rear and right side perspective view of a lock for use in the delivery tool of the present invention;

FIG. 36 is a rear elevation view of the lock of FIG. 35;

FIG. 37 is a side elevation view of the lock of FIG. 35;

FIG. 38 is a top, rear and right side perspective view of slider mechanism for use in the delivery tool of the present invention;

FIG. 39 is a top plan view of the slider mechanism of FIG. 38;

FIG. 40 is a left side elevation view of the slider mechanism of FIG. 38 with the opposite side being substantially identical;

FIG. 41 is a bottom plan view of the slider mechanism of FIG. 38;

FIG. 42 is a rear elevation view of the slider mechanism of FIG. 38;

FIG. 43 is a front elevation view of the slider mechanism of FIG. 38;

FIG. 44 is an enlarged top elevation view of a latching mechanism of the slider mechanism of FIG. 38;

FIG. 45 is a top, rear and right side perspective view of transport lock for use in the delivery tool of the present invention; and

FIG. 46 is a side elevation view of the transport lock of FIG. 45.

### V. DESCRIPTION OF THE PREFERRED EMBODIMENT

With reference now to the various drawing figures, in which identical elements are numbered identically throughout, a detailed description of a preferred embodiment of the present invention will now be provided. The teachings of the following U.S. patents are incorporated herein by reference: U.S. Pat. No. 6,250,307 to Conrad et al. dated Jun. 26, 2001; U.S. Pat. No. 6,578,580 to Conrad et al. dated Jun. 17, 2003; U.S. Pat. No. 6,523,542 to Metzger et al. dated Feb. 25, 2003; U.S. Pat. No. 6,513,530 to Knudson et al. dated Feb. 4, 2003; and U.S. Pat. No. 6,431,174 to Knudson et al. dated Aug. 13, 2002.

A. Physiology Background

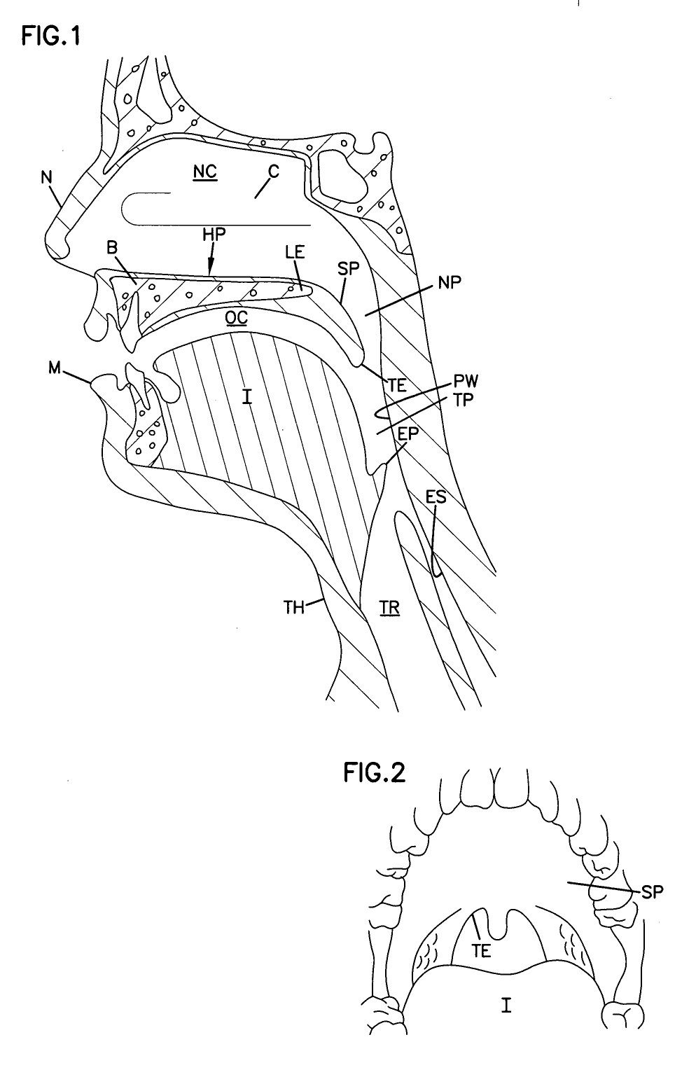

FIG. 1 shows, in cross-section, a naso-pharyngeal area of an untreated patient. FIG. 2 shows a soft palate SP viewed through an open mouth of the untreated patient. FIG. 1 shows the nose N, mouth M and throat TH. The tongue T is shown in an oral cavity OC of the mouth. A hard palate HP (containing a bone B) separates the oral cavity OC from the nasal cavity NC. The nasal concha C (soft tissue which defines, in part, the nasal sinus—not shown) resides in the nasal cavity NC.

The soft palate SP (a muscle activated soft tissue not supported by bone) depends in cantilevered manner at a leading end LE from the hard palate HP and terminates at a trailing end TE. Below the soft palate SP, the pharyngeal wall PW defines the throat passage TP. A nasal passage NP connects the nasal cavity NC to the pharyngeal wall PW. Below an epiglottis EP, the throat passage TP divides into a trachea TR for passing air to the lungs and an esophagus ES for passing food and drink to the stomach.

The soft palate SP is operated by muscles (not separately shown and labeled) to lift the soft palate SP to urge the trailing edge TE against the rear area of the pharyngeal wall PW. This seals the nasal cavity NC from the oral cavity OC during swallowing. The epiglottis EP closes the trachea TR during swallowing and drinking and opens for breathing.

For purposes of this disclosure, the nasal cavity NC, oral cavity OC and throat passage TP are collectively referred to as the naso-pharyngeal area (or airway) of the patient with the area including the various body surfaces which cooperate to define the nasal cavity NC, oral cavity OC and throat passage TP. These body surfaces include outer surfaces of the nasal concha C, the upper and lower surfaces of the soft palate SP and outer surfaces of the pharyngeal wall PW. Outer surfaces means surfaces exposed to air. Both the upper and lower surfaces of the soft palate SP are outer surfaces.

Snoring can result from vibration of any one of a number of surfaces or structures of the naso-pharyngeal area. Most commonly, snoring is attributable to vibration of the soft palate SP. However, vibratory action of the nasal concha C and the pharyngeal wall PW can also contribute to snoring sounds. It is not uncommon for vibratory action from more than one region of the naso-pharyngeal area to contribute to snoring sounds. Sleep apnea can result from partial or full collapse of the naso-pharyngeal wall during sleep as well as having nasal and palatal contributions.

As indicated above, most of the present discussion will describe placing a stiffening implant in the soft palate SP, it will be appreciated the present invention is applicable to other regions of the naso-pharyngeal area including the nasal concha C and the pharyngeal wall PW. Also, it will be appreciated the present invention is applicable to airway conditions such as OSA or snoring and is not intended to be limited to snoring although this indication will be most frequently referenced for purpose of illustration of the invention. It will also be appreciated the present invention can be used with different types of implants (i.e., any of those referenced in the references incorporated by reference above) or any other implant which may be delivered from a needle.

The snoring sound is generated by impulses caused by rapid obstruction and opening of airways. Huang, et al., state the airway passage opening and closing occurs 50 times per second during a snore. Huang, et al., utilize a spring-mass model (FIG. 5) to illustrate oscillation of the soft palate in response to airflow (where the soft palate is the ball B of mass depending by a spring S from a fixed anchor A).

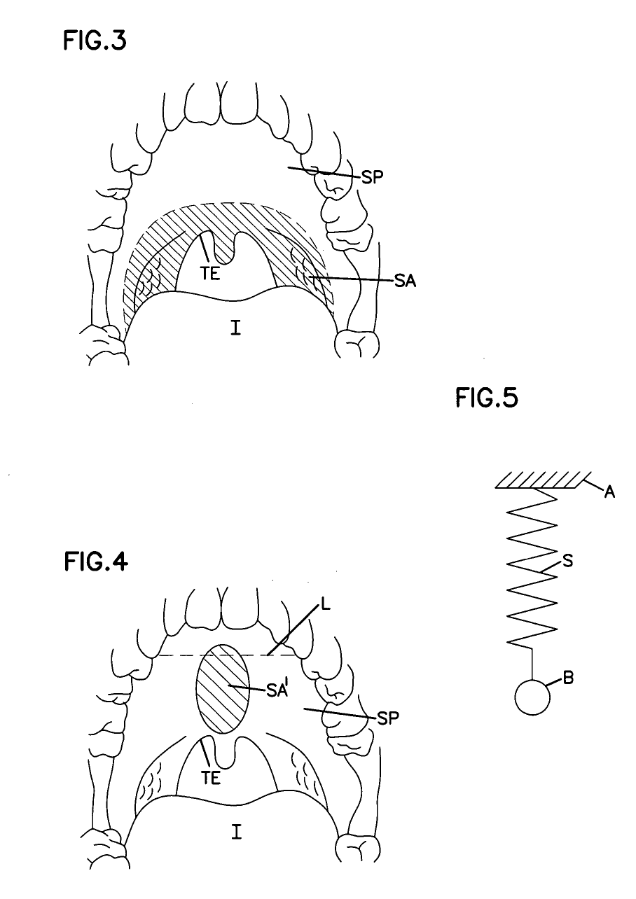

A prior art technique for treating the soft palate is uvulopalatopharyngoplasty (UPPP). In UPPP, a trailing edge of the soft palate is removed. The shaded area SA in FIG. 3 shows the area of the trailing end TE of the soft palate SP to be removed during this procedure. Huang, et al., analogize the shortening of the soft palate SP in UPPP as effectively raising the critical airflow speed at which soft palate flutter will occur. An alternative procedure proposed by Huang, et al., reduces the flexibility of the soft palate SP through surface scarring which is asserted as affecting the critical flow speed. The shaded area SA′ in FIG. 4 shows the area to be scarred by this alternate procedure. In FIG. 4, dashed line L shows the demarcation between the soft and hard palates.

Using the spring-mass model of FIG. 5 as a convenient model of the soft palate SP, the present invention is directed to a delivery system for a surgical implant for the soft palate SP to alter the elements of the model and thereby alter the dynamic response of the soft palate SP to airflow.

B. Disclosure of Commonly Assigned U.S. Patents

The aforementioned commonly assigned US patents (which have been incorporated herein by reference) describe a wide variety of airway implants for treating snoring or OSA. These patents are U.S. Pat. Nos. 6,250,307; 6,578,580; 6,523,542; 6,513,530 and 6,431,174.

In a presently preferred embodiment, the implant 20 is a braid of fibers 22. While a single type fiber could be used in implant 20, the implant can be formed of two or more different fibers braided or twisted together. For example, one fiber may be provided for encouraging fibrotic response. Such a fiber may be polyester or silk suture material. The other fiber may be a bio-resorbable fiber (e.g., bio-resorbable suture material which may include natural materials such as collagen or synthetic materials such as the PDS suture material). Alternatively, the other fiber may be a non-resorbable material such as polypropylene suture material to provide added stiffness to the implant.

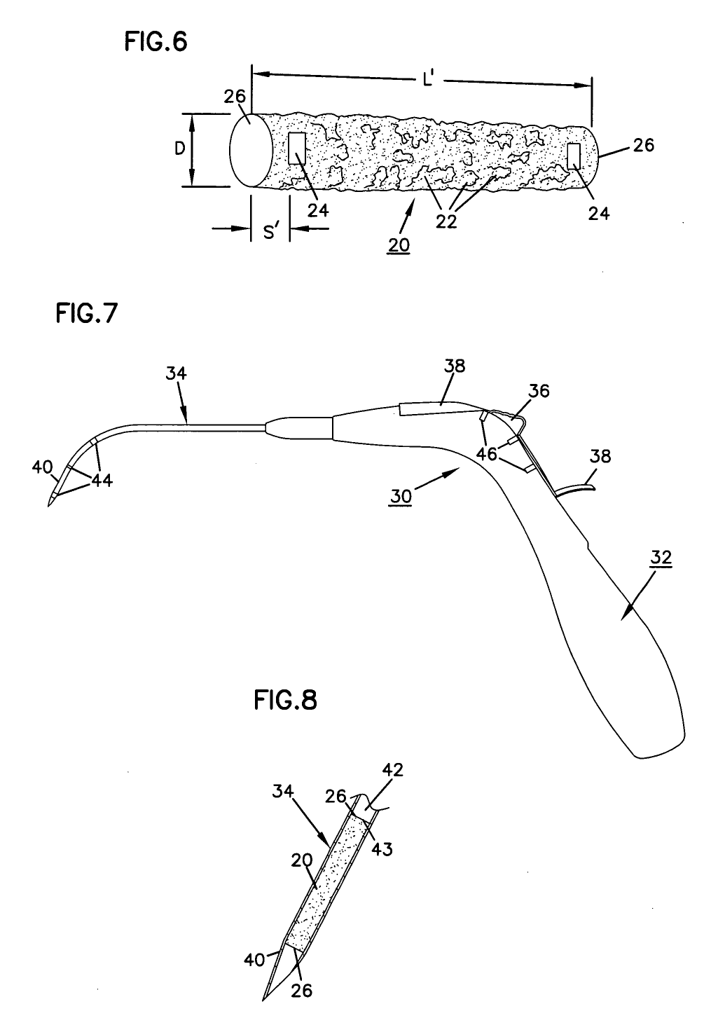

In a preferred embodiment (shown in FIG. 6), the implant 20 is a composite braid of both air-textured and non-air-textured yarns of polyester formed in a braid of about 2 mm in diameter (D) and 18 mm in length (L′). Welds 24 are formed near the ends 26 of the implant 20 to bond the fibers 22. The welds 24 are spaced from the ends 26 by a spacing S′ so that the fibers 22 in the spacing are free to fray and present a fluffier area for tissue in-growth. The implant 20 is fibrosis inducing to induce a fibrotic response of tissue following implantation. An implant having the foregoing characteristics is more fully described in the aforementioned U.S. Pat. No. 6,513,530.

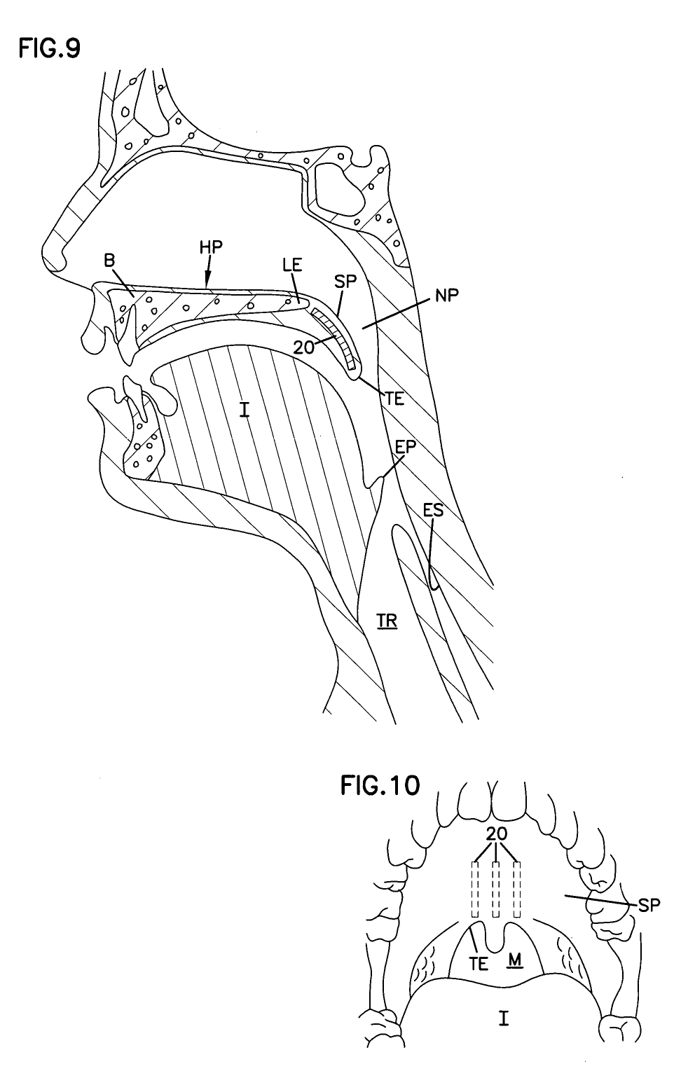

FIGS. 7 and 8 show a prior delivery tool 30 for placing the implant 20 in the soft palate SP. FIGS. 9 and 10 illustrate the desired placement of the implant 20 in the soft palate SP. As shown in FIG. 10, three implants are preferably placed in the soft palate SP. One at the soft palate midline and one each on opposite sides of the midline about 5 mm from the midline.

The delivery tool 30 includes a handle 32 and a needle 34 permanently secured to the handle 32. The handle 32 is designed to be hand-grasped in a pistol-grip manner with a sliding thumb switch 36 positioned to oppose the operator's thumb (not shown) when the handle 32 is grasped. A removable tape 38 covers the thumb switch 36 during shipping and storage to prevent undesired movement of the thumb switch 36. The distal tip 40 of the needle 34 is bent to permit ease of placement of the tip 40 in the soft palate SP without interference of the tool 30 with the patient's teeth or hard palate.

The needle 34 is connected to the thumb switch 36 through linkage (not shown) contained within the handle 32 such that the needle retracts to the right (i.e., moves rearward into the handle 32) as the thumb switch is slid downwardly on the handle 32. Directions “right”, “rearward” and “down” are with reference to the orientation shown in FIG. 7.

The distal tip 40 of needle 34 has a bevel ground for piercing tissue of the soft palate. The needle 34 is hollow and carries the implant 20 in sliding close tolerance at the distal tip 40.

An obturator or rod 42 is positioned in the needle 34 between the implant 20 and the handle 32. The obturator 42 is secured to the handle 32 so that is does not retract as the needle retracts. Therefore, as the needle 34 retracts, the needle 34 slides over the fixed-place obturator 42. The distal end 43 of the obturator 42 butts against the implant 20. This prevents the implant 20 from moving with the needle 34 as the needle 34 retracts. As a result, the retracting needle 34 exposes the implant 20.

The implant 20 is carried by the needle 34 to a desired implant site within the soft palate SP. At the desired site, the implant 20 is deployed by retracting the needle 34. Retraction is performed by retracting back on thumb switch 36. Retraction of the needle relative to the handle causes the obturator 42 to dispel the implant 20 from the needle 34. The aforementioned U.S. Pat. No. 6,578,580 describes a retracting needle and stationary rod to deploy an implant in the soft palate. That patent also describes a pre-loaded implant.

The needle has markings 44 to provide indication to an operator of depth of penetration of the needle tip 40 in tissue and location of the implant 20. Corresponding markings 46 are placed on the handle 32 to illustrate the degree of needle retraction and implant exposure relative to sliding movement of the thumb switch 36.

The implant 20 is pre-loaded into the needle 34. The needle 34 is non-removably secured to the handle 32 and all components are delivered in a sterile package. Such packaged implant and its delivery system are the subject of 510k clearance (K011723) from the U.S. Food and Drug Administration and CE Mark certification (CE 66447) from BSI Product Services indicated for use with the treatment of socially disruptive snoring and 510k clearance (K040417) from the U.S. Food and Drug Administration indicated for use with the treatment of sleep apnea. The product is sold by Restore Medical Inc., St. Paul, Minn., USA—assignee of the present application.

C. Apparatus of the Present Invention

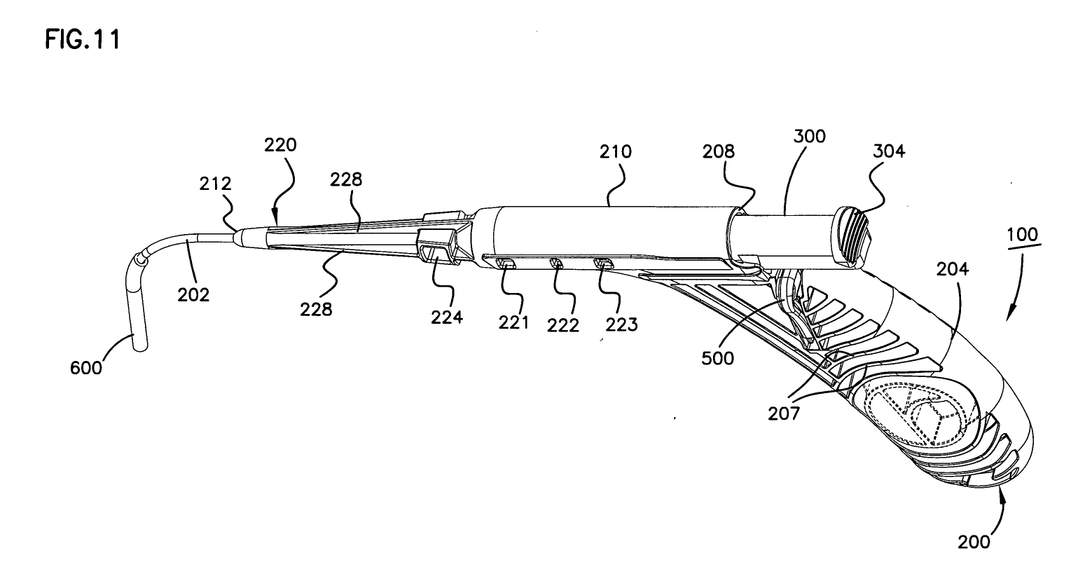

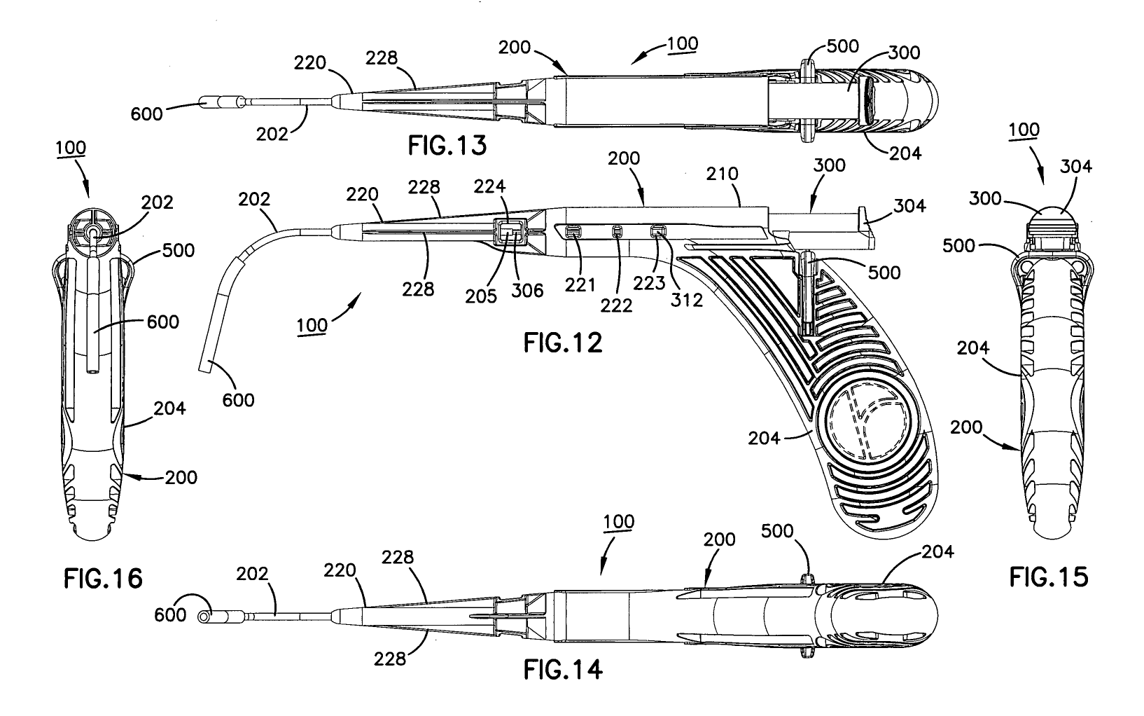

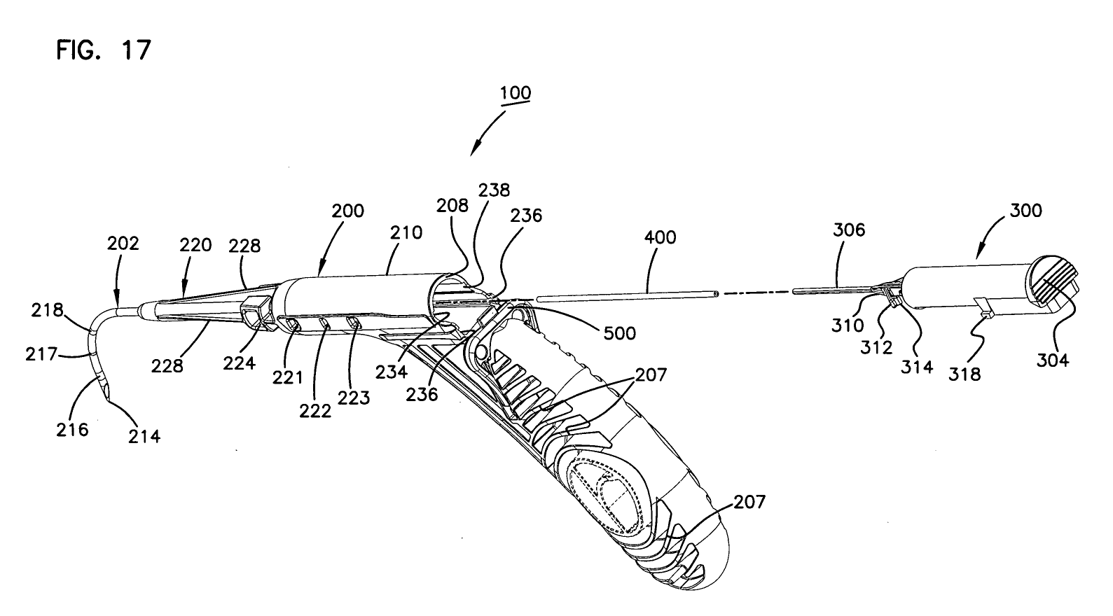

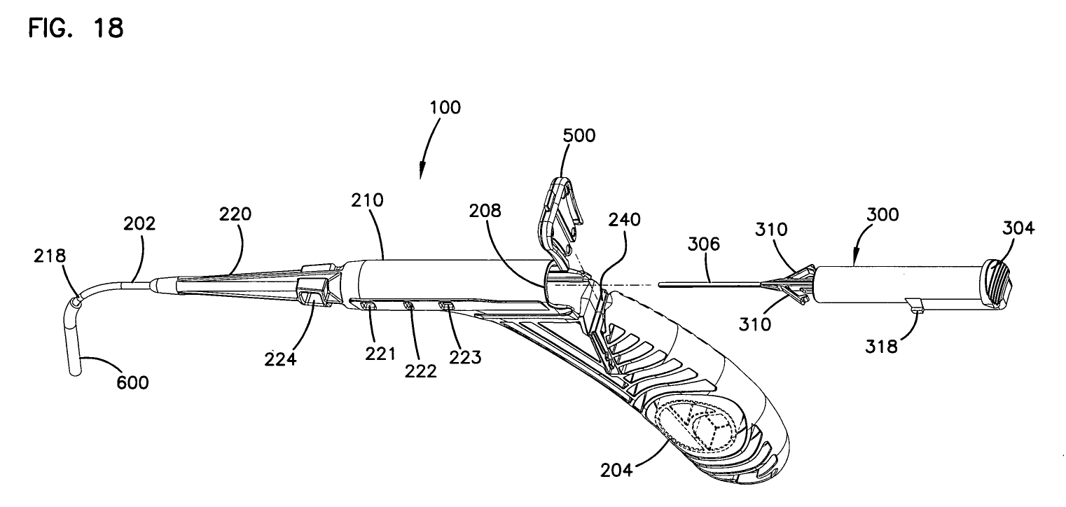

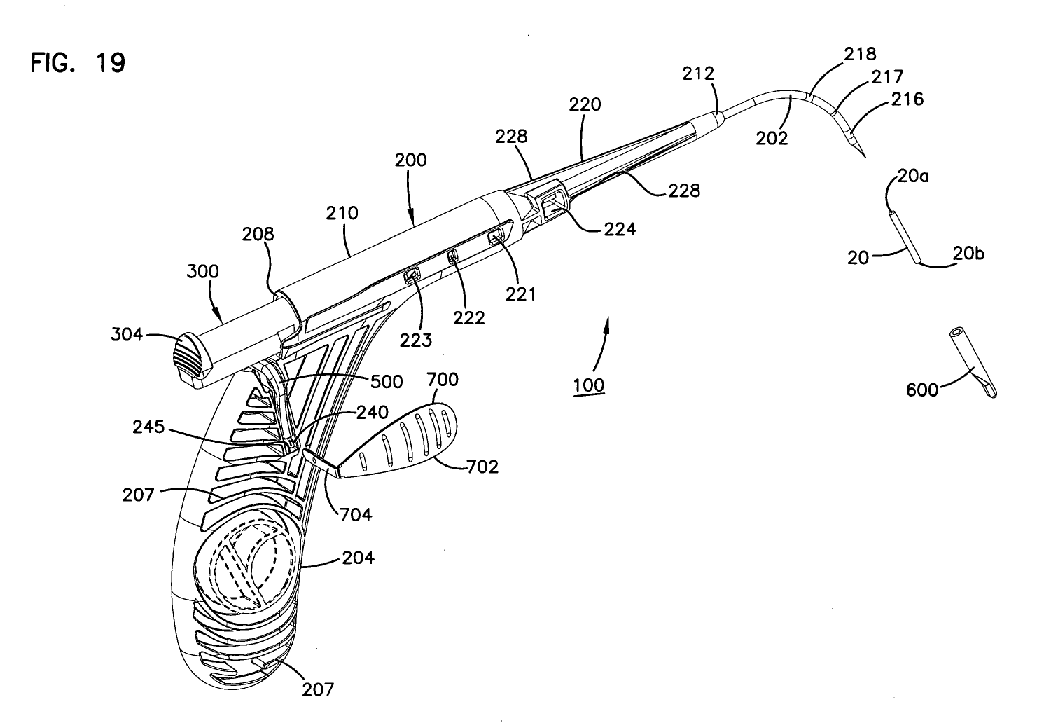

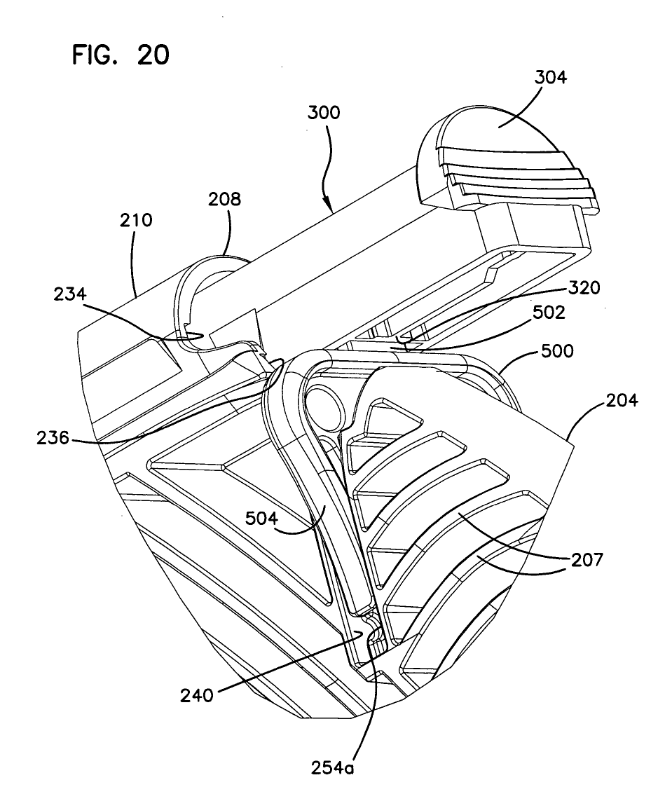

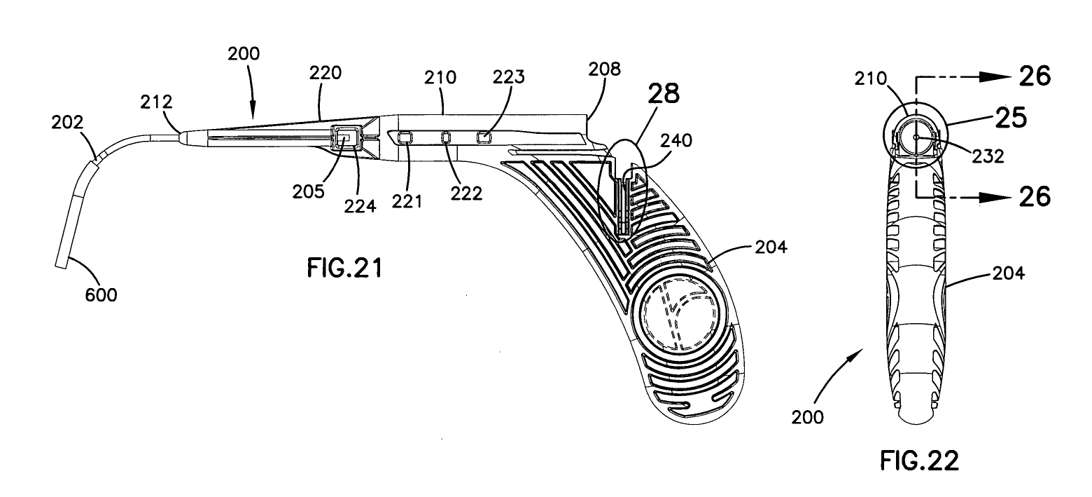

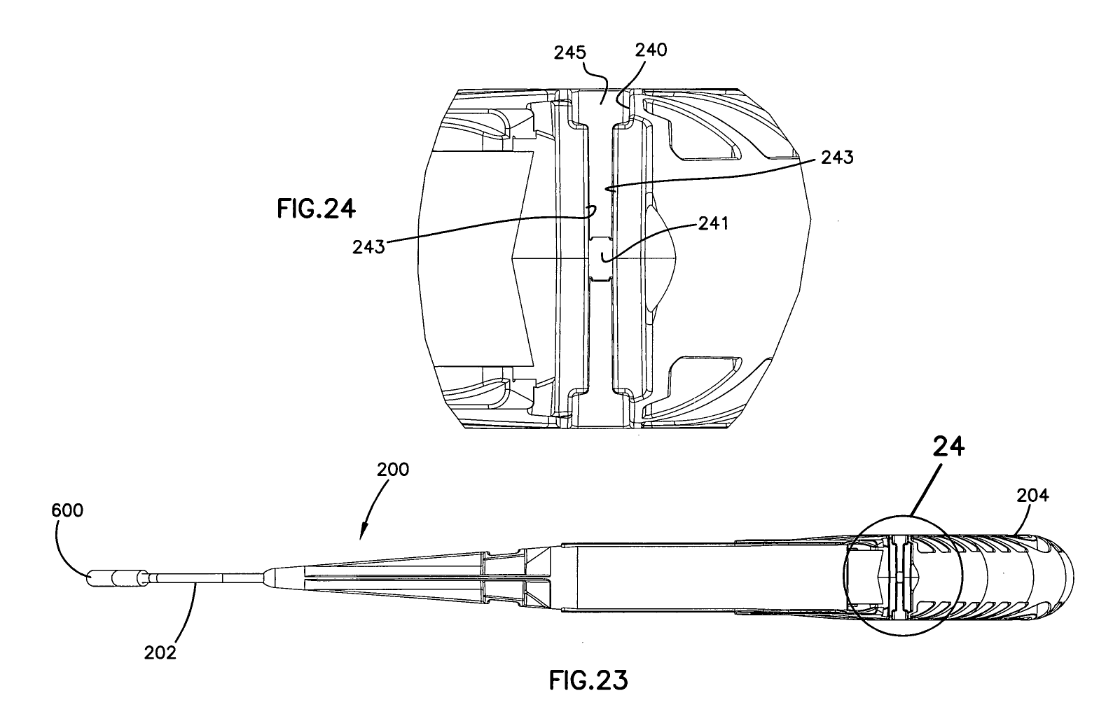

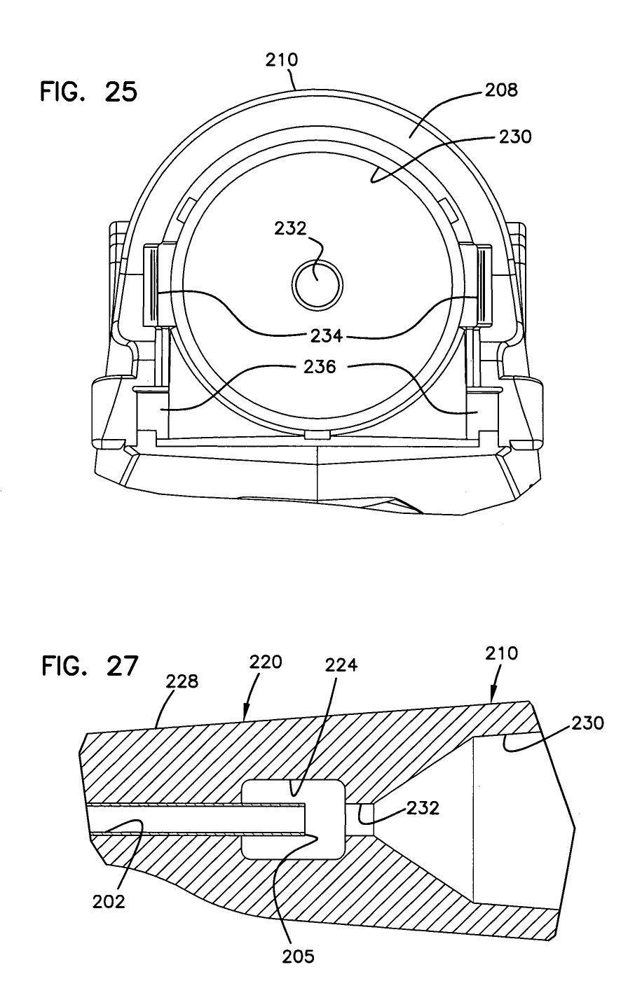

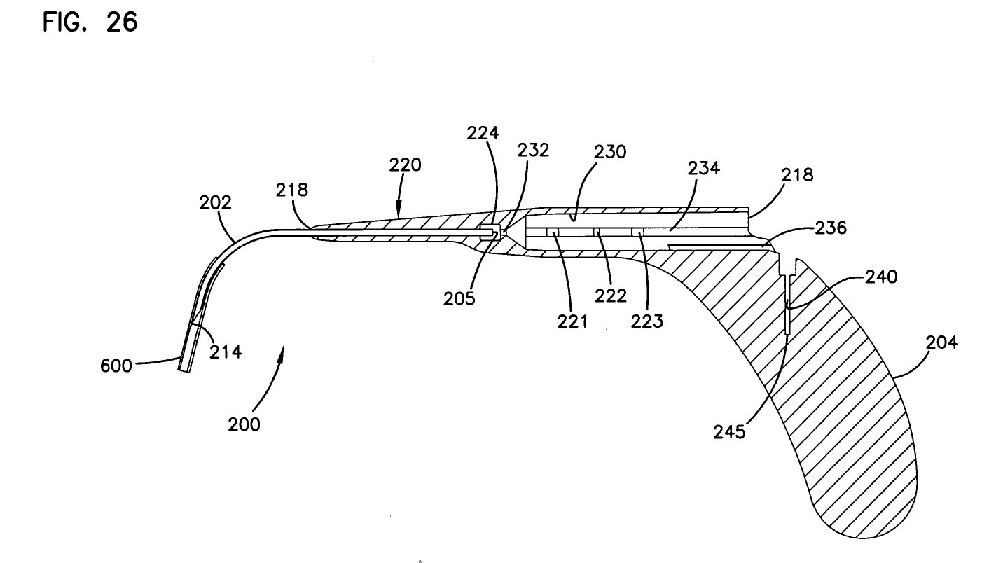

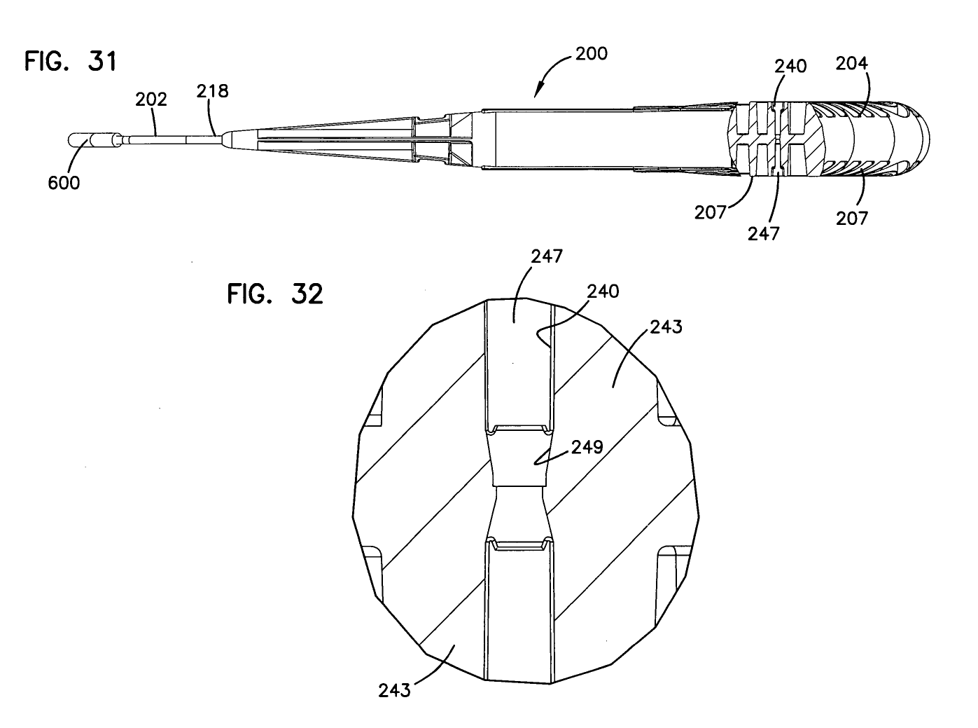

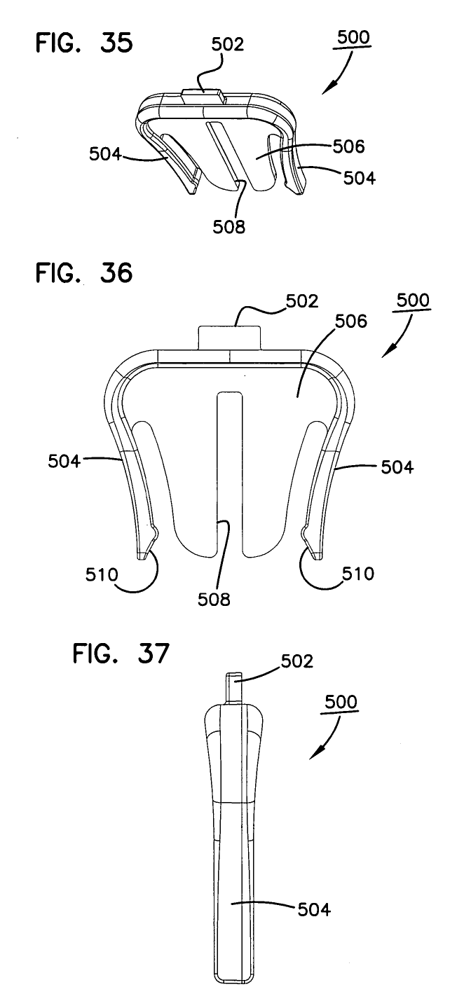

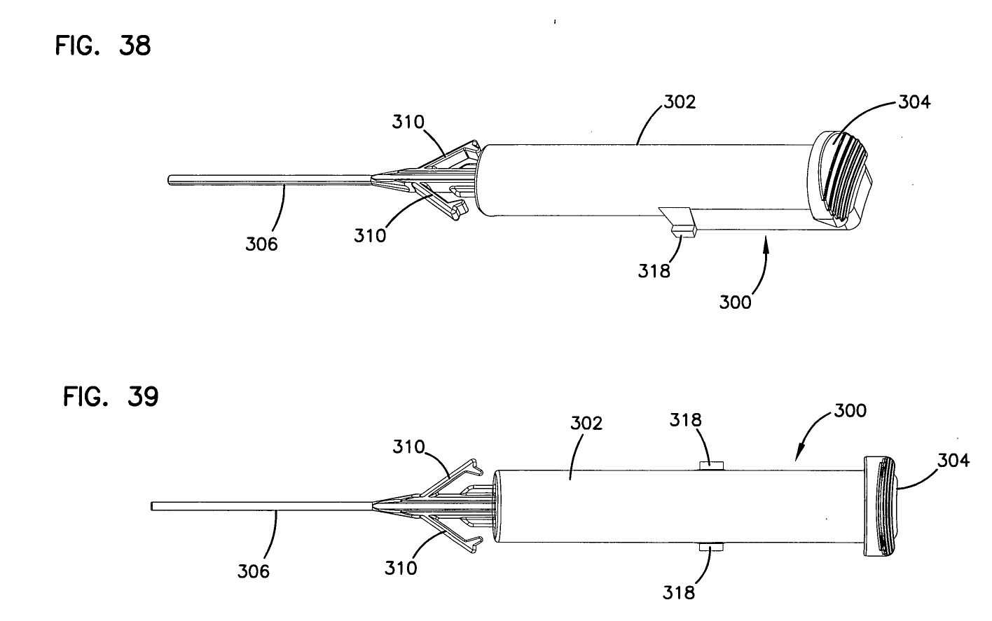

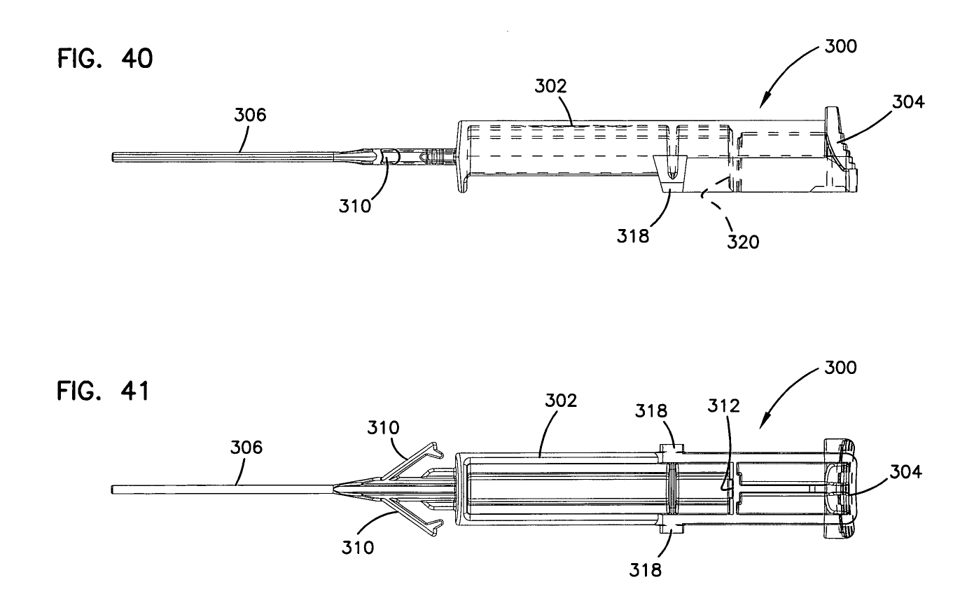

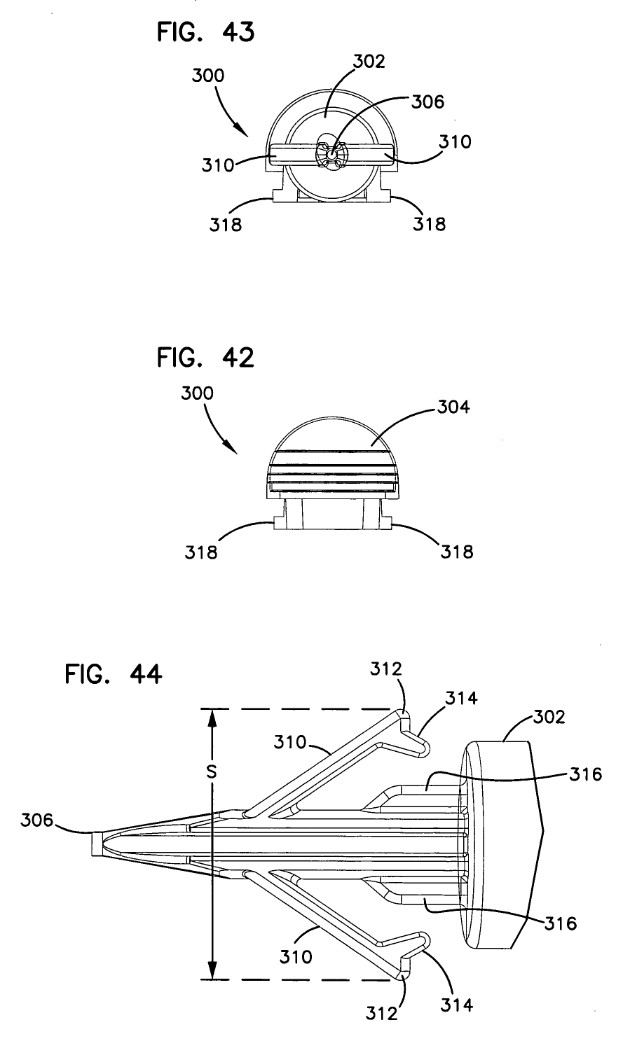

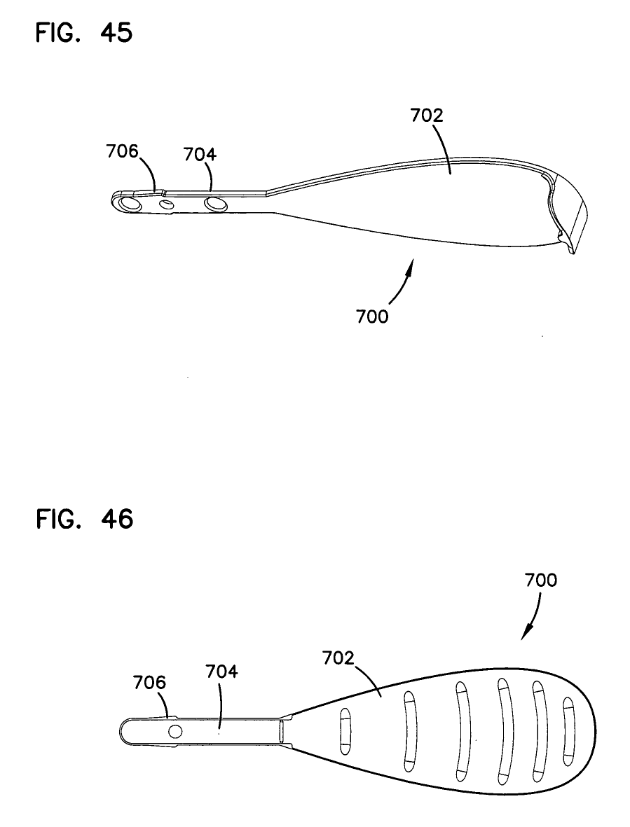

With reference to FIGS. 11-16, the present invention is shown in a preferred embodiment. As shown in FIGS. 17, 18 and 19, the delivery system 100 of the present invention includes a handle/needle sub-assembly 200 (separately shown in FIGS. 21-32), a slider mechanism 300 (separately shown in FIGS. 38-44), an obturator 400 (shown only in FIG. 17), a lock 500 (shown separately in FIGS. 35-37) an implant 20 (FIG. 19), a needle safety cap 600 (FIG. 19) and a transport lock 700 (shown separately in FIGS. 45 and 46).

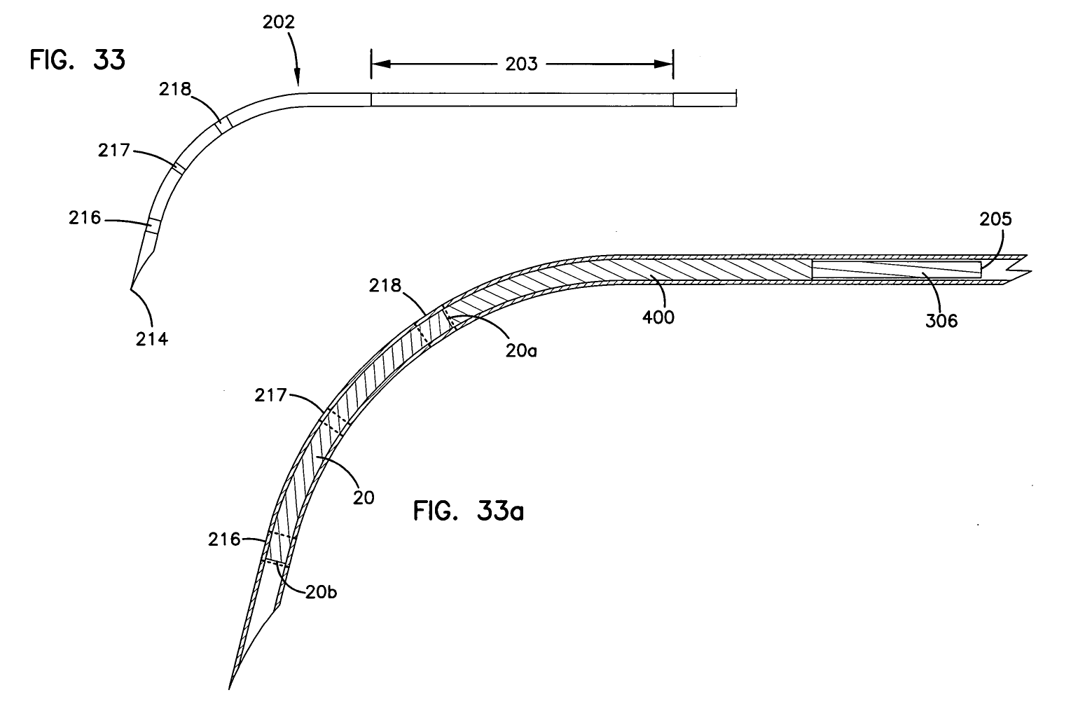

The implant 20 is carried in a distal end of the needle 202. Since, in a preferred embodiment, the implant 20 is identical to that shown in FIG. 6 it is identically numbered in the views of FIGS. 19 and 33A.

As described above, the implant 20 is a braid of fibrosis-inducing fibers extending prom a proximal end 20a to a distal end 20b. The implant 20 may have a diameter slightly larger than the interior diameter of the needle 202 such that the implant 20 may expand upon ejection from the needle 202. As described in the aforementioned patents (e.g., U.S. Pat. No. 6,250,307), the implant 20 is adapted to alter a dynamic response of airway tissue following placement of the implant in the tissue.

Needle/Handle Sub-Assembly

The needle/handle sub-assembly 200 is separately shown in FIGS. 21-32. The handle-needle sub-assembly 200 includes a hollow needle 202 permanently molded to the a 204 in an injection plastic molding process. The molding process will be separately described.

The handle 204 is pistol-shaped and includes a grip 206 sized to be received within the palm of a physician and with the physician's thumb opposing an opening 208 of a barrel 210. The barrel 210 extends from the handle 204 and connects with an axially aligned needle retention portion 220 which terminates at a distal end 212.

The needle 202 projects out of the distal end 212 and curves downwardly to a needle distal tip 214. A plurality of markings are formed on the needle. These include a distal marking 216, an intermediate marking 217 and a proximal marking 218. The distance between the distal marking 216 and proximal marking 218 is approximate to the length of the implant 20 and correspond with an approximate positioning of the implant 20 within the interior of the hollow needle 202. As shown in FIG. 33A, when the implant 20 is in the needle 202, implant ends 20a, 20b are generally aligned with markings 218, 216, respectively.

The curvature of the needle 202 is selected to permit a physician to insert the needle tip 214 into the tissue of the soft palate while angling the delivery system 100 at an appropriate angle to permit visualization within the patient's mouth and to avoid interference from the patient's teeth. The markings 216-218 permit a visual indication of the relative depth of insertion of the needle 202 into the patient's tissue.

As will be more fully described, the handle 204 is formed from injection molded plastic. The needle 202 has a length 203 which is textured as illustrated in FIG. 33. The textured length 203 is spaced a distance from a proximal end 205 of the needle 202. The length 203 is sized to be approximate the length of the needle retention portion 220 of the handle 204.

The barrel 210 includes three openings 221, 222, and 223 on opposite sides of the barrel 210 and spaced along its axial dimension. As will be more fully described, the openings 221-223 will cooperate with the slider mechanism 300 to provide a visual, audible and tactile indication to a physician of the degree of ejection of the implant 20 from the needle 202.

As will become apparent, the distal, intermediate and proximal openings 221, 222, and 223 correspond with the distal, intermediate and proximal markings 216, 217, and 218 on the needle 202. When slider mechanism 300 is engaged with the proximal openings 223, the implant 20 is fully inserted within the needle 202 (in the positioning of FIG. 33A). At this position, a proximal end 20a of the implant 20 is aligned with the proximal marking 218. When the slider mechanism 300 is engaged with the intermediate opening 222, the proximal end 20a of the implant 20 is passing the intermediate marker 217. When the slider mechanism 300 is engaged with the distal opening 221, the proximal end 20a of the implant 20 is passing the distal marker 216.

Intermediate of the barrel 210 and the needle retention portion 220 is an opening 224. The proximal end 205 of the needle 202 is exposed within the opening 224. As will become apparent, the placement of the proximal end 205 of the needle 202 within the opening 224 permits bonding of the needle 202 to the handle 204 during the plastic injection molding process while avoiding a flow of plastic into the needle 202.

The obturator 400 is a flexible plastic rod which is fully inserted within the needle 202 abutting the proximal end 20a of the implant 20 (as best shown in FIG. 33A). As will be more fully described, movement of the slider mechanism 300 within the barrel 210 results in urging the obturator 400 to urge the implant 20 out of the needle 202.

The sides of handle grip 206 includes a plurality of ribs 207. Formation of the ribs 207 permits uniform distribution of plastic in the molding process. Further, the ribs 207 produce a gripping surface to ensure the physician has a sure grip on the handle 204.

A plurality of support ribs 228 extend along the length of both sides and top of the needle retention portion 220 to add structural rigidity to the needle retention portion 220. A support rib is not formed on the lower surface to avoid interference of such a support rib with the teeth of the patient during placement. The tapered geometry of the ribs 228 provides an enhanced line of sight for the physician to view the target area in the tissue in which the implant 20 is to be placed.

The barrel 210 has an internal cavity 230 extending along its axial length. The cavity 230 is tapered to a central distal hole 232 extending into the opening 224.

A first set of longitudinally extending side recesses 234 (see, e.g., FIGS. 25 and 26) extend parallel to the axis of the cavity 230 and on opposite sides of the cavity 230. The first side recesses 234 are in line with the openings 221-223. A second set of side recesses 236 extend along opposite sides of the internal barrel cavity 230 parallel to and spaced from the first side recesses 234.

Slider Mechanism

The slider mechanism 300 (separately shown in FIGS. 38-44) has a generally cylindrical body 302 sized to be slidably received within the barrel cavity 230. A proximal end of the body 302 has a thumb tab 304 positioned to be engaged by the thumb of the physician whose palm is gripping the handle 204.

A pin 306 extends axially away from a distal end of the body 302 and is sized to pass through the distal hole 232 and into the opening 224. Further, the pin 306 is sized to be passed into the proximal end 205 of the needle 202 and oppose the obturator 400 within the needle 202 when the slider mechanism 300 is mounted within the barrel cavity 230.

Rearward extending lever arms 310 extend from the pin 306 toward the distal end of the body 302 and are flared outwardly to terminate at ends 312. The ends 312 have a rest spacing S (shown in FIG. 44) greater than a diameter of the body 302 and greater than the diameter of the barrel cavity 230.

The lever arms 310 are resilient such that they will bend to permit insertion of the lever arms 310 into the smaller diameter barrel cavity 230. At ends 312, stops 314 are formed opposing ribs 316 (FIG. 44) to limit an amount of inward deflection of the lever arms 310 toward the axis of the body 302 and thereby preventing breakage of the lever arms 310 from excessive bending.

The lever arms 310 are sized and positioned to ride in the first side recesses 234. Outwardly projecting tabs 318 are formed on the body 302 and sized and positioned to ride within the second side recesses 236. Accordingly, the slider mechanism 300 will axially slide within the barrel cavity 230 with the pin 306 sliding within the needle 202.

During assembly, the slider mechanism 300 is sized and configured such that when the needle pin 306 is abutting the obturator 400 (which in turn is abutting the proximal end of the implant 20) within the needle 202 (as shown in FIG. 33A), the ends 312 of the arms 310 oppose the proximal side openings 223. The resilient bias of the lever arms 310 causes the lever arms 310 to spread outwardly at the openings 223 for the ends 312 to be captured within the openings 223. On forward movement (i.e., movement of slider 300 toward the distal end 212), the angled arms 310 act as cam followers against the plastic surrounding the openings 221-223 which urges the ends 312 inwardly to clear the openings. In reverse movement, there is no corresponding cam follower and the ends 312 and stops 314 block such motion.

The rearward angled arms 310 permit forward movement of the slider mechanism 300 within the cavity 230 by block rearward movement. The slider mechanism 300 cannot be slid rearward out of the barrel cavity 230 since the arms 310 oppose the plastic material defining the openings 223. To retract the slider mechanism 300 from this position, the lever arms 310 would have to be urged inwardly such that the ends 312 clear the openings 323. This would be inconvenient for the physician. This inconvenience for retracting the slider mechanism 300 has operational benefits as will be described.

The positioning of the ends 312 within the openings 223 presents a visual indication that the delivery device 100 is in a first position to initiate ejection of the implant from the needle 202. This is also a preferred position for the delivery device 100 to be in during storage or transportation.

As an operator grips the handle 204 and presses a thumb against end 304, the slider mechanism 300 is urged forward within the barrel cavity 230. Such advancement continues until the ends 312 of the lever arms 310 oppose the intermediate side openings 222 at which point the natural resilience of the lever arms 310 causes the ends 312 to snap into the opposed openings 222.

The snapping action provides creates both a tactile and audible indication to a physician that their intermediate positions corresponding with openings 222 have been attained. These are in addition to the visible indication of the ends 312 residing in the openings 222. This advises the physician that the proximal end 20a of the implant 20 has been displaced such that the proximal end 20a is now generally aligned with the intermediate marking 217 on the needle 202. Again, the slide mechanism 300 cannot readily be retracted from this position.

Upon further advancement of the slide mechanism 300 within the barrel cavity 230, the ends 312 of the lever arms 310 are aligned with the side openings 221. At this positioning, the ends 312 snap into positioning within the openings 221 providing an indication (both tactile, audible and visual) that the slide mechanism 300 has been fully inserted into the barrel cavity 230 and that the proximal end 20a of the implant 20 is now generally aligned with the distal marking 216 of the needle 202 which is associated with full deployment of the implant 20 from the needle 202. Again, without use of a special tool, the slide mechanism 300 may not be retracted.

The inability to retract the slide mechanism 300 without use of a special tool to depress the lever arms 310 provides significant advantages for both safety and operation. In a common application, three implants 20 are to be placed within a soft palate using separate delivery systems 100. The implant 20 is not readily observable within the needle 202. Once a delivery tool 100 is used, the slide mechanism 300 is fully inserted in the barrel cavity 230. Maintaining a used delivery tool 100 in a state with the slide mechanism 300 fully inserted prevents a physician from mistakenly retracting the slide mechanism 300 of a used device 100 and believing that the apparatus is in fact a loaded device with a loaded implant 20 ready for use. This prevents unnecessary puncture wounds in the patient and avoids potential for transmitting pathogens from used needles 202.

The use of both visual, audible and tactile indications as described permits a physician to rely upon any of these three indicators while placing an implant 20 in the soft tissue. Therefore, the physician is free to maintain visual focus on the implant site within the tissue and notice the positioning of the needle markings 216-218 relative to the tissue while placing the implant 20 within the tissue.

Molding Process

The apparatus 100 thus described has very few parts. This results in reduced cost and reduced manufacturing effort as well as reduced opportunities for error during assembly procedures.

The needle 202 is molded into the handle 204. The texturing of the needle 202 along length 203 ensures that when the needle 202 is molded with the handle 204, the needle 202 is bonded to the plastic of the handle 204 and cannot move axially or rotate.

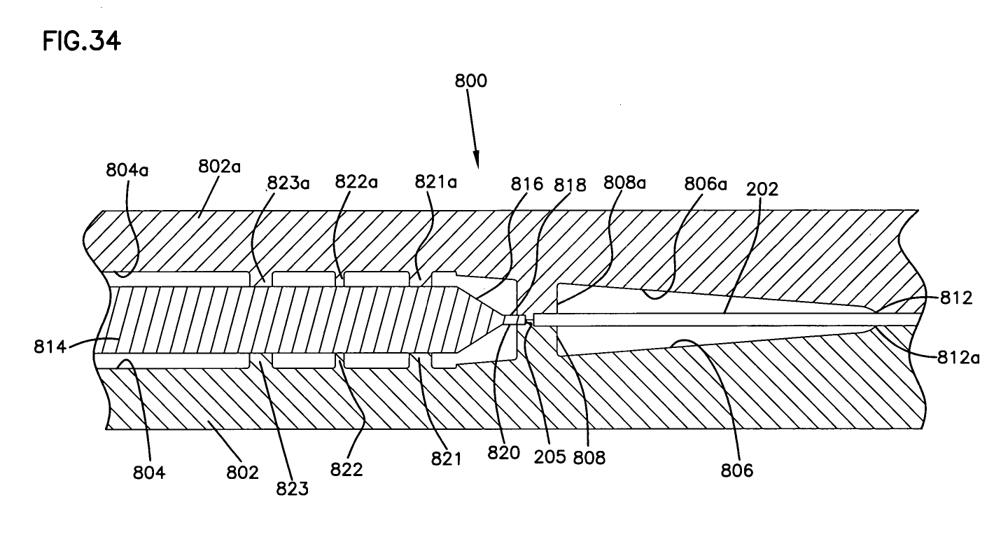

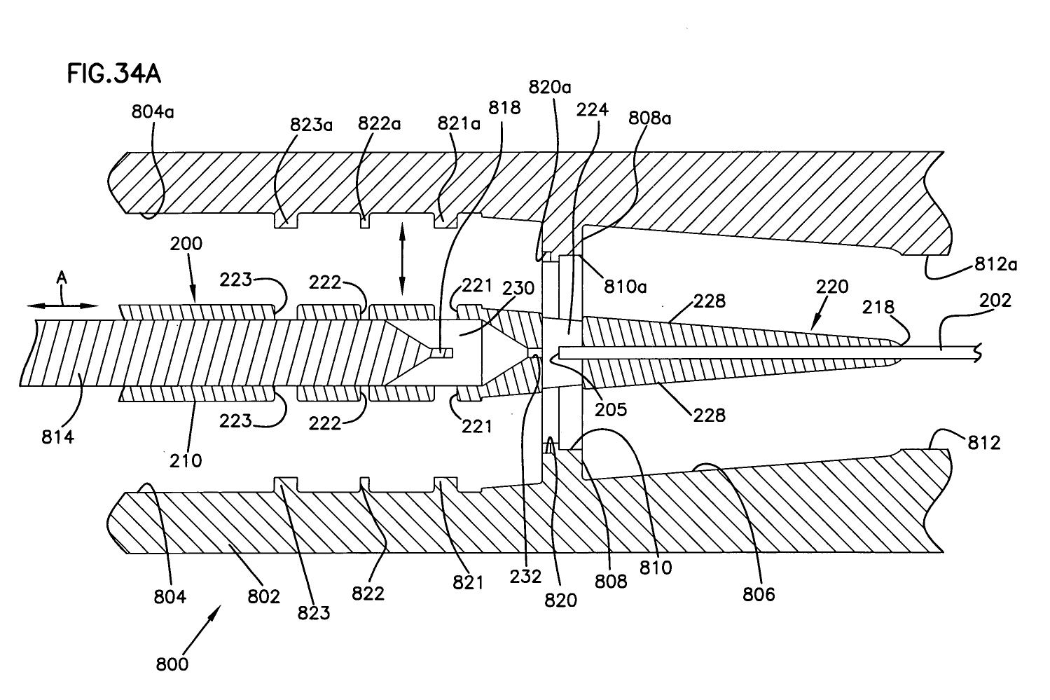

FIGS. 34 and 34A illustrate the molding process. The process forms a part-line on the handle centrally positioned between the right and left sides of the handle 204.

Shown schematically in FIGS. 34, 34A, the mold 800 includes a right side 802 for forming the impressions on the right side of the handle 204 and a left side 802a for forming impressions on the left side of the handle 204. The sides 802, 802a are substantially identical to form substantially identical handle sides.

The mold halves 802, 802a include barrel defining surfaces 804, 804a and needle detention portion defining surfaces 806, 806a. The barrel defining surfaces 804, 804a and needle retention portion defining surfaces 806, 806a are separated by protruding blocks 808, 808a that form the opening 224. The blocks 808, 808a include recesses 810, 810a sized to receive and hold the proximal end 205 of the needle 202. A distal blocks 812, 812a join to hold the needle 202 at the distal end 212 of the needle retention portion 220. If desired, the blocks 808, 808a and 812, 812a can be provided with vacuum ports to securely position the needle 202 within the blocks 808, 808a and 812, 812a during the molding process.

A mold pin 814 is positioned between the surfaces 804, 804a to create the void of the barrel cavity 230. A leading end 816 of the pin 814 is tapered create the tapered distal end of the barrel cavity 230 with a protruding pin 818 positioned to be received within a complimentary shaped hole 820 formed in blocks 808, 808a to create hole 232 and to ensure accurate alignment of the barrel cavity 230 within the barrel 210. The barrel cavity defining surfaces 804, 804a have inwardly protruding pins 821-823 and 821a-823a to form the side openings 221, 222, 223.

With the needle 202 placed within the blocks 808, 808a and 812, 812a and with the pin 814 fully inserted within the mold, plastic is injected into the voids defined between the opposing surfaces of the mold components to form the handle 204 on the needle 202. The blocks 808, 808a cover the proximal end 205 of needle 202 preventing plastic from flowing into the proximal end of the needle. Upon completion of the injection molding, the pin 814 moves axially out of the barrel cavity 230 (in the direction of arrow A in FIG. 34A) and the mold halves 802, 802a separate transversely (illustrated by the arrow B in FIG. 34B) to reveal a completed handle/needle sub-assembly 200. In practice, only one mold half 802, 802a need move with the sub-assembly 200 ejected from the other mold half by ejector pins (not shown).

Lock Mechanism

It is desirable that when the slider mechanism 300 is in its initial position (with ends 312 in openings 223), the slider mechanism 300 should be locked from further advancement into the barrel cavity 230 until use of the delivery device 100 is imminent. To achieve this, the lock 500 is provided.

As shown in FIGS. 24, 28 and 29, a transverse slot 240 is formed through the grip 206 rearward of the barrel opening 208. The slot has a bottom wall 247. The lock 500 is a plastic sheet which slidably moves up and down within the slot 240.

The slot 240 has a rib 241 separating the slot 240 into left and right sides. The slot also has front and rear plates 243 with upper and lower detents 245, 245a.

The lower portion of the slider mechanism 300 is exposed and includes a transverse stop surface 320. The lock includes a stop tab 502. A stop surface 320 is positioned such that the stop tab 502 opposes the stop surface 320 with the stop tab 502 positioned between the surface 320 and the barrel opening 208 when the slide mechanism 300 is in the position with the ends 312 received within the proximal openings 223. Accordingly, in this position the stop tab 502 prevents the slide mechanism from moving forward within the barrel cavity 230.

The lock 500 can be slid downwardly within the slot 240 to release the tab 502 from opposing the stop surface 220 thereby permitting an operator to urge the slide mechanism 300 to move forward in the barrel cavity 230. The lock 500 has a plate 506 sized to pass between plates 243. The plate 506 has a slot 508 to receive the rib 241. The lock 500 has resilient prongs 504 with a thickness sized to be received within slot 240 but wider than the spacing between the plates 243. The prongs 504 have barbed ends 510 opposing detents 322 formed in the slot 240. The detents 242 receive the prongs 204 to permit the stop to be releasably held in either a lock position (with barbs 510 in upper detents 245) or an unlock position (with barbs 510 in lower detents 245a).

In the locked position, the upper tab 502 protrudes into the hollow body of the slider mechanism 300 and opposes wall 320 to block forward movement of the slider mechanism 300. In this position, the ends 312 in holes 223 prevent rearward movement. In the unlocked position, the tab 502 is removed from the body of the slider mechanism 300 permitting its forward movement.

Transport Lock

In transportation, it is desirable that the needle tip 214 be protected and that the lock 500 be held in place in the locked position and not be permitted to move downwardly to the unlocked position. A plastic safety cap 600 is provided to slidably fit over the needle distal tip 214 to prevent exposure of the sharp tip until the safety cap 600 is removed from the needle distal tip 214. A transport lock 700 is provided to hold the lock 500 in the locked position.

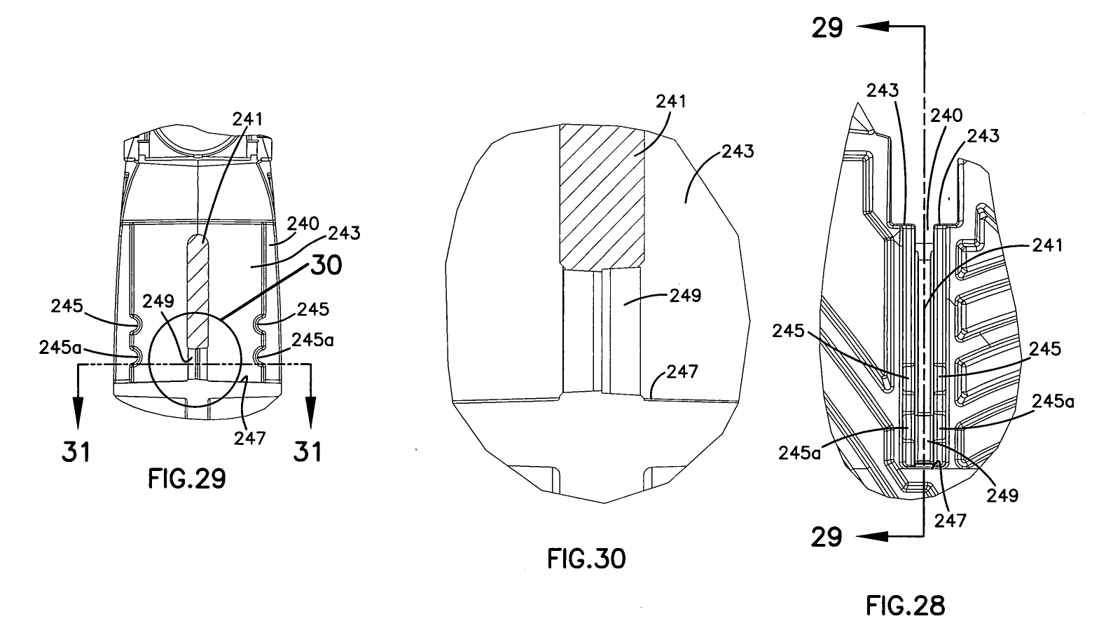

The transport lock 700 with a handle 702 and a tab 704. The tab 704 is sized to be slidably received within the slot 240. The tab 704 is sized to slide with the narrow slot 249 beneath rib 241 (FIG. 28). So positioned, the tab 704 is between the bottom of the lock 500 and the bottom 247 of the slot 240 when the lock 500 is in the locked position. Accordingly, the transport lock 700 prevents the lock 500 from sliding downward to the unlocked position during handling or transportation. The rib 241 is spaced from the slot bottom 247 to define a transverse passage 249 narrower than tab 704. The end of the tab 700 has a flexible enlarged tip 706 to releasably retain the lock 700 in place.

When it is desired to use the delivery apparatus, the safety cap 600 is removed and the transport lock 700 is removed. A physician is then free to move the lock 500 downward to the unlocked position and then advance the slide mechanism 300 into the barrel cavity 230. During the sliding action, the pin 306 urges the obturator 400 to urge the implant out of the distal tip of the needle.

The present invention has been described in a first preferred embodiment for delivery of a braided implant into tissue of the soft palate to treat an airway condition such as obstructive sleep apnea or socially disruptive snoring. It will be appreciated the present invention covers a wide variety of implants (e.g., instead of the braided implant, the implant may be a bolus of particulate material as described in U.S. Pat. No. 6,431,174 or a sheet of fibrosis-inducing material as described in U.S. Pat. No. 6,523,542). Also, the invention is readily adapted through change of needle size and curvature to permit placement of an implant in airway tissue other than the soft palate (e.g., placement in tissue of the nasal cavity or pharyngeal wall).

1. An apparatus for use in treating an airway condition of a patient, said apparatus comprising:
a handle sized to be hand-grasped by an operator;
a barrel connected to said handle and having a barrel cavity accessible through a barrel opening, said cavity extending axially from said opening to a distal end;
a needle retention portion positioned distal to said barrel;
a needle having a distal tip for penetrating into said tissue, said needle having an axially extending bore, said needle retained within said needle retention portion and with a proximal end of said needle opposing said distal end of said barrel;
an implant of biocompatible material sized to be embedded within a tissue of said airway, said implant disposed within said needle bore at said distal tip;
an actuator disposed within said barrel cavity and movable in a deployment direction from said barrel opening toward said distal end of said barrel cavity, said actuator adapted to urge said implant from said distal tip as said actuator is moved in said deployment direction;
an indicator indicating a relative positioning of said actuator within said barrel cavity.

2. An apparatus according to claim 1 comprising a stop for inhibiting movement of said actuator in a direction opposite said deployment direction.

3. An apparatus according to claim 1 comprising a lock for releasably locking said actuator in a first position within said barrel cavity.

4. An apparatus according to claim 1 further comprising:
an obturator disposed for slideable movement within said bore of said needle;
said actuator including a driver positioned to move said obturator toward said implant upon movement of said actuator in said deployment direction.

5. An apparatus according to claim 1 wherein said needle is bonded to said needle retention portion.

6. An apparatus according to claim 1 wherein said implant is adapted to alter a dynamic response of said tissue following placement of said implant in said tissue.

7. An apparatus according to claim 1 wherein said implant includes a material for promoting tissue in-growth into said implant following placement of said implant into said tissue.

8. An apparatus according to claim 1 wherein said implant is sized slightly greater than said bore for said implant to expand upon ejection from said bore.

9. An apparatus according to claim 8 wherein said implant is formed of multiple fibers including fibers of said material for promoting tissue in-growth.

10. An apparatus according to claim 9 wherein the multiple fibers are twisted together along a length of the implant with the fibers having terminal ends at opposite ends of the implant.

11. An apparatus according to claim 10 wherein the multiple fibers are braided together.

12. An apparatus according to claim 1 wherein the handle includes a pistol grip having a textured gripping surface.

13. An apparatus according to claim 12 wherein the textured gripping surface includes a plurality of generally parallel ribs.
